(19) United States
(12) Patent Application Publication
(10) Pub. No.: US 2007/0275988 A1
(43) Pub. Date: Nov. 29, 2007

(54) Transition state structure and inhibitors of thymidine phosphorylases

(76) Inventor: Vern L. Schramm, New Rochelle, NY (US)

Correspondence Address: AMSTER, ROTHSTEIN & EBENSTEIN LLP, 90 PARK AVENUE, NEW YORK, NY, 10016, US

(21) Appl. No.: 10/570,475

(22) PCT Filed: Sep. 3, 2004
(86) PCT No.: PCT/US04/28703

§ 371 Date: Nov. 1, 2006

Related U.S. Application Data
(60) Provisional application No. 60/500,847, filed on Sep. 5, 2003.

(51) Int. Cl. A61K 31/513; A61K 31/427; C07D 417/02; C07D 403/02

(52) U.S. Cl. 514/269; 514/365; 544/310; 548/200

(57) ABSTRACT

The transition state structure of thymidine phosphorylase is provided, along with thymidine phosphorylase inhibitors that resemble the charge and geometry of the thymidine phosphorylase transition state. Also provided are methods of inhibiting a thymidine phosphorylase, methods of treating cancer, and methods of inhibiting angiogenesis which utilize the thymidine phosphorylase transition state inhibitors.

### CROSS-REFERENCE TO RELATED APPLICATIONS

This application claims the benefit of U.S. Provisional Application No. 60/500,847, filed Sep. 5, 2003.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of Grant GM41916 awarded by the National Institutes of Health.

### BACKGROUND OF THE INVENTION

(1) Field of the Invention

The present invention generally relates to enzyme inhibitors. More specifically, the invention relates to the transition state structure of thymidine phosphorylase and its use as a blueprint for the design of transition state analog inhibitors of thymidine phosphorylases, the transition state analog inhibitors of thymidine phosphorylase, and methods of using those inhibitors.

(2) Description of the Related Art

### REFERENCES CITED

Aoki T et al. (2002) Correlation between malignancy grade and p53 in relation to thymidine phosphorylase activity in colorectal cancer patients. Oncol. Rep. 9(6): 1267-71.

Bainbridge J W et al. (2003) Gene therapy for ocular angiogenesis. Clin. Sci. (Lond) 104(6):561-75.

Brown N S, Bicknell R. (1998) Thymidine phosphorylase, 2-deoxy-D-ribose and angiogenesis. Biochemical Journal 334(Pt 1): 1-8.

Burke-Gaffney A et al. (2002) Regulation of chemokine expression in atherosclerosis. Vascul. Pharmacol. 38(5):283-92.

Cole C et al. (1999) A similarity model for the human angiogenic factor, thymidine phosphorylase/platelet derived-endothelial cell growth factor. Anticancer Drug Des. 14(5):411-20.

Crandall D L et al. (1997) A review of the microcirculation of adipose tissue: anatomic, metabolic, and angiogenic perspectives. Microcirculation 4(2):211-32.

Gudas L J, Ullman B, Cohen A, Martin D W Jr. (1978) Deoxyguanosine toxicity in a mouse T lymphoma: relationship to purine nucleoside phosphorylase-associated immune dysfunction. Cell 14(3):531-8.

Hotchkiss K A et al. (2003) Mechanisms by which tumor cells and monocytes expressing the angiogenic factor thymidine phosphorylase mediate human endothelial cell migration. Cancer Res. 63(2):527-33.

Igawa H et al. (2003) Influence of platelet-derived entothelial cell growth factor/thymidine phosphorylase on the cell cycle in head and neck squamous cell carcinoma in vitro. Oncol. Rep. 10(4):967-71.

Kicska G A et al. (2001) Immucillin H, a powerful transition-state analog inhibitor of purine nucleoside phosphorylase, selectively inhibits human T lymphocytes. Proc. Natl. Acad. Sci. USA 98(8):4593-8.

Moghaddam A, Zhang H T, Fan T P, Hu D E, Lees V C, Turley H, Fox S B, Gatter K C, Harris A L, Bicknell R. (1995) Thymidine phosphorylase is angiogenic and promotes tumor growth. Proc. Natl. Acad. Sci. USA. 92(4):998-1002.

Paleolog E M (2002) Angiogenesis in rheumatoid arthritis. Arthritis Res. 4(suppl 3):S81-S90.

Reynolds L P et al. (2002) Angiogenesis in the female reproductive organs: pathological implications. Int. J. Exp. Pathol. 83(4):151-63.

Reynolds K, Farzaneh F, Collins W P, Campbell S, Bourne T H, Lawton F, Moghaddam A, Harris A L, Bicknell R. (1994) Association of ovarian malignancy with expression of platelet-derived endothelial cell growth factor. Journal of the National Cancer Institute. 86(16): 1234-8.

Sato J et al. (2003) Role of thymidine phosphorylase on invasiveness and metastasis in lung adenocarcinoma. Int. J. Cancer 106(6):863-70.

Takao S, Aidyama S I, Nakajo A, Yoh H, Kitazono M, Natgugoe S, Mivadera K, Fukushime M, Yamada Y, Aikou T. (2000) Suppression of metastasis by thymidine phosphorylase inhibitor. Cancer Research. 60(19):5345-8.

Taylor R N et al. (2002) Angiogenic factors in endometriosis. Ann. NY Acad. Sci. 955:89-100.

Tokunaga Y et al. (2002) Prognostic value of thymidine phosphorylase/platelet-derived endothelial cell growth factor in advanced colorectal cancer after surgery: evaluation with a new monoclonal antibody. Surgery 131(5):541.

Tsukagoshi S et al. (2003) Thymidine phosphorylase-mediated angiogenesis regulated by thymidine phosphorylase inhibitor in human ovarian cancer cells in vivo. Int. J. Oncol. 22(5):961-7.

Nucleoside phosphorylases play an integral role in several types of human cancers. For example, purine nucleoside phosphorylase (PNP) is well known to be essential for the proliferation of T-cells in lymphocytic leukemias (Gudas et al., 1978). More recently, thymidine phosphorylase (aka platelet derived-endothelial cell growth factor; PD-ECGF) has been implicated as a significant contributor to angiogenesis in many solid tumor types (Reynolds et al., 1994). TP expression has been shown to correlate with the aggressiveness and invasiveness of several different types of human tumors, including breast and colon primary cell carcinomas (Moghaddam et al., 1995; Aoki et al., 2002). Additionally, the most potent inhibitor of TP, (TPI-FIG. 7) (Kᵢ=20 nM), has been shown to have some antiangiogenic activity in vivo (Takao et al., 2000).

Thymidine phosphorylase (TP) catalyzes the reversible phosphorolysis of the glycosidic bond of thymidine forming thymine and 2-deoxyribose 1-phosphate (FIG. 1) which is subsequently metabolized to 2-deoxyribose. It is believed that 2-deoxyribose is eventually responsible for the angiogenic effects of TP (Brown & Bicknell, 1998). TP also promotes endothelial cell migration in vitro, also apparently due to 2-deoxyribose (Hotchkiss et al., 2003).

In addition to its role in thymidine metabolism and in angiogenesis, TP is the primary enzyme of fluoropyrimidine antimetabolite (for example 5-fluoro-2′-deoxyuridine [FdUrd]) degradation. The fluoropyrimidines are one of the most frequently used chemotherapeutic drugs for the treatment of GI malignancies, including colorectal cancer. One approach used clinically to treat liver metastases arising from colorectal cancer is the hepatic artery infusion of FdUrd. FdUrd is converted to its active metabolite, FdUMP, by thymidine kinase. The opposing reaction, catalyzed by TP, converts FdUrd to 5-fluorouracil (FUra). Since FUra is 100 to 1000-fold less potent than FdUrd, the metabolism of FdUrd by TP tends to reduce the activity of the anticancer drug. TP inhibitors would thus be useful clinically when used in combination with FrUrd to potentiate its antitumor activity, particularly in more aggressive tumors that express high levels of TP.

In addition to cancer, inappropriate induction of angiogenesis plays an important role in other diseases, for example rheumatoid arthritis (Paleolog, 2002), retinal diseases such as diabetic retinopathy and age related macular degeneration (Bainbridge et al., 2003), atherosclerosis (Burke-Gaffnet et al., 2002), various diseases of female reproductive organs such as endometriosis (Reynolds et al., 2002; Taylor et al., 2002), and obesity (Crandall et al., 1997).

Thus, there is a need for improved inhibitors of TP. The present invention addresses that need.

### SUMMARY OF THE INVENTION

Accordingly, the inventors have established the transition state structure of thymidine phosphorylase. The transition state structure provides a blueprint to design thymidine phosphorylase inhibitors that resemble that transition state structure. These inhibitors are useful, for example, in inhibiting angiogenesis and in cancer treatments.

Thus, in some embodiments, the invention is directed to transition state inhibitors of a thymidine phosphorylase. In these embodiments, the transition state inhibitors are compounds that resemble the charge and geometry of the thymidine phosphorylase transition state.

In other embodiments, the invention is directed to methods of inhibiting a thymidine phosphorylase. The methods comprise combining the thymidine phosphorylase with any of the transition state inhibitors described above.

Additionally, the invention is directed to methods of treating cancer in a mammal. The methods comprise administering to the mammal any of the transition state inhibitors described above.

The invention is also directed to methods of inhibiting angiogenesis in a mammal. These methods also comprise administering to the mammal any of the transition state inhibitors described above.

The invention is additionally directed to methods of designing an inhibitor of thymidine phosphorylase. The methods comprise designing a compound that resembles the charge and geometry of the thymidine phosphorylase transition state.

### BRIEF DESCRIPTION OF THE DRAWINGS

FIG. 1 provides the thymidine phosphorylase reaction.

FIG. 2 provides preferred examples of thymidine phosphorylase inhibitors that are transition state analogs.

FIG. 3 provides a schematic of chemical methods for synthesizing radiolabeled thymidine. Panel a. Synthesis of ATP from glucose. Panel b. Synthesis of thymidine from ATP. Compounds shown in boldface type are purified. Abbreviations used are: AD, adenosine deaminase; AK, adenylate kinase; AP, alkaline phosphatase; APRT, adenosine phosphoribosyl transferase; G6PDH, G6P dehydrogenase; 6PGDH, 6-phosphogluconate dehydrogenase; HK, hexokinase; PK, pyruvate kinase; PNP, purine nucleoside phosphorylase; PPase, inorganic pyrophosphatase; PRI, phosphoriboisomerase; PRPS, 5-ribosyl-1-pyrophosphate synthesis; RTR, ribonucleotide triphosphate reductase; TP, thymidine pbosphorylase.

FIG. 4 shows a graph of experimental results of the arsenolysis of thymidine by TP.

FIG. 5 shows the intrinsic KIEs of thymidine.

FIG. 6 shows the experimentally derived model of the TP transition state structure.

FIG. 7 shows examples of compounds subjected to electron potential mapping and determination of ability to inhibit thymidine phosphorylase.

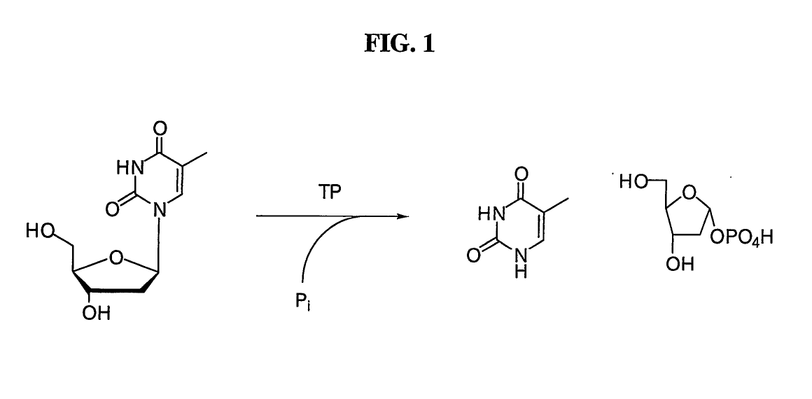

### DETAILED DESCRIPTION OF THE INVENTION

The present invention is based on the discovery of the transition-state structure of thymidine phosphorylase (TP). The experimental results leading to the discovery of the transition-state structure is described in Example 1.

The discovery of the TP transition-state structure allows the design of transition-state analogs that would be expected to be strong inhibitors of TP. This is established by studies of a related enzyme, purine nucleoside phosphorylase (PNP), showing that transition-state analogs having a similar charge and geometry as the transition state compound of an enzyme are likely to be strong inhibitors of that enzyme (Kicska et al. 2001).

The transition state of TP, provided in Example 1 below, has a surprising structure, since there is apparently little oxacarbenium charge buildup, whereas the transition state was expected to be an oxacarbenium ion (Cole et al., 1999).

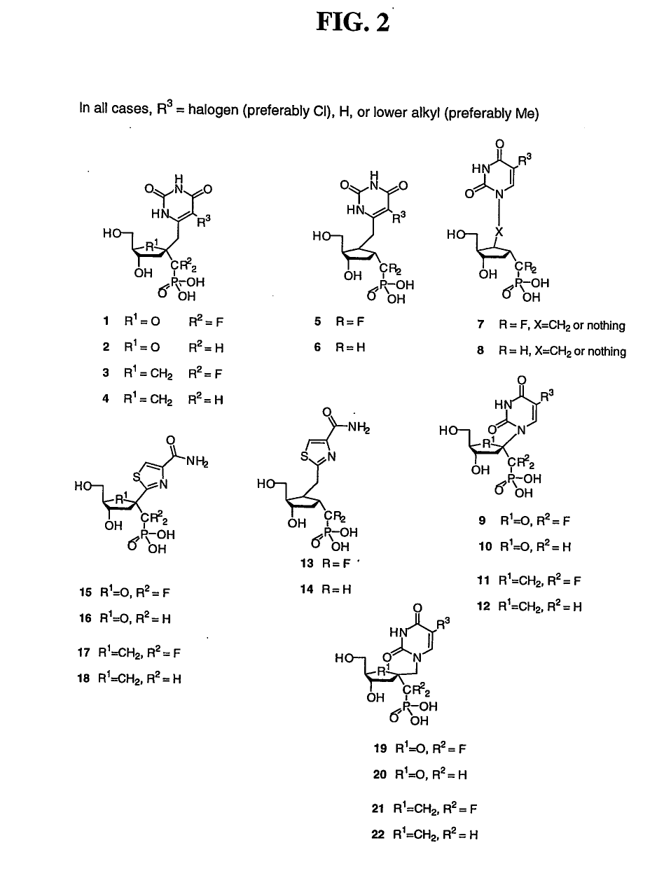

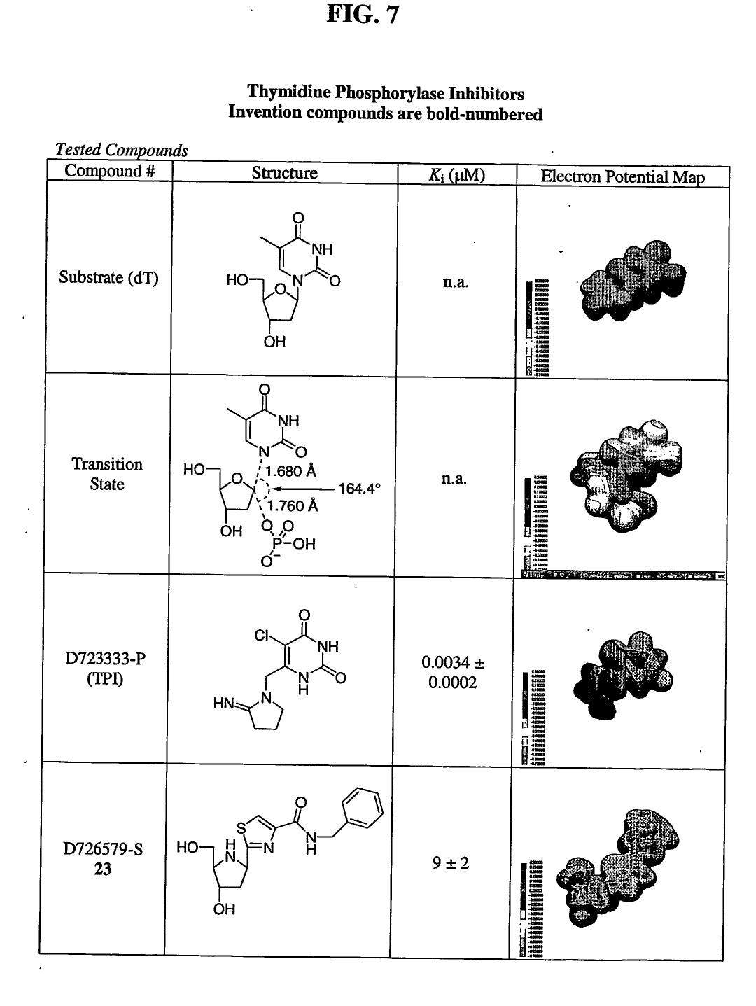

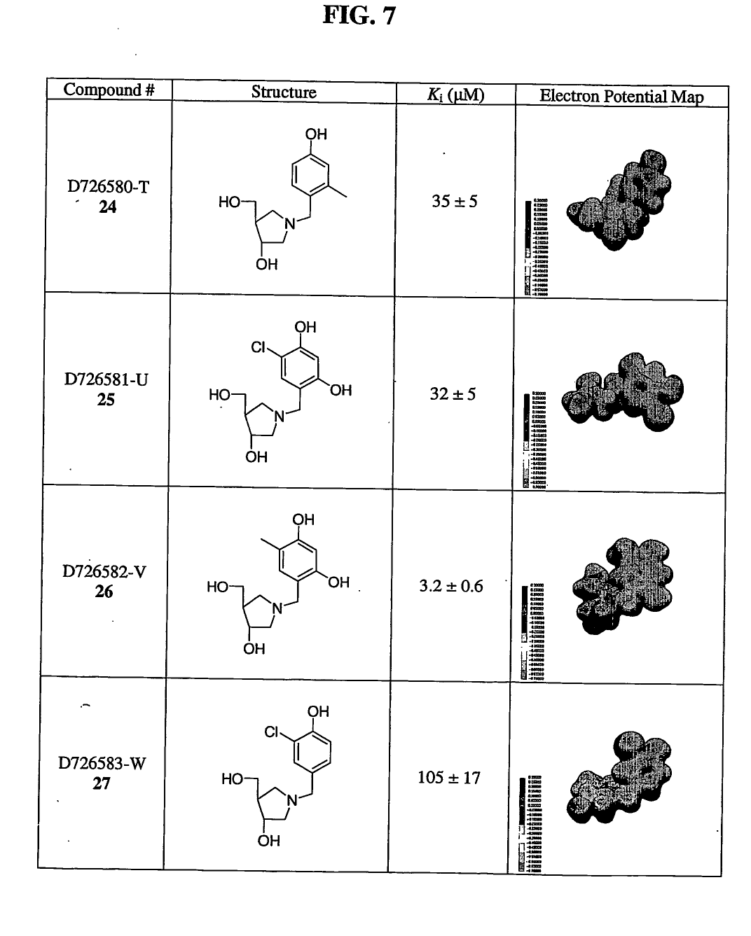

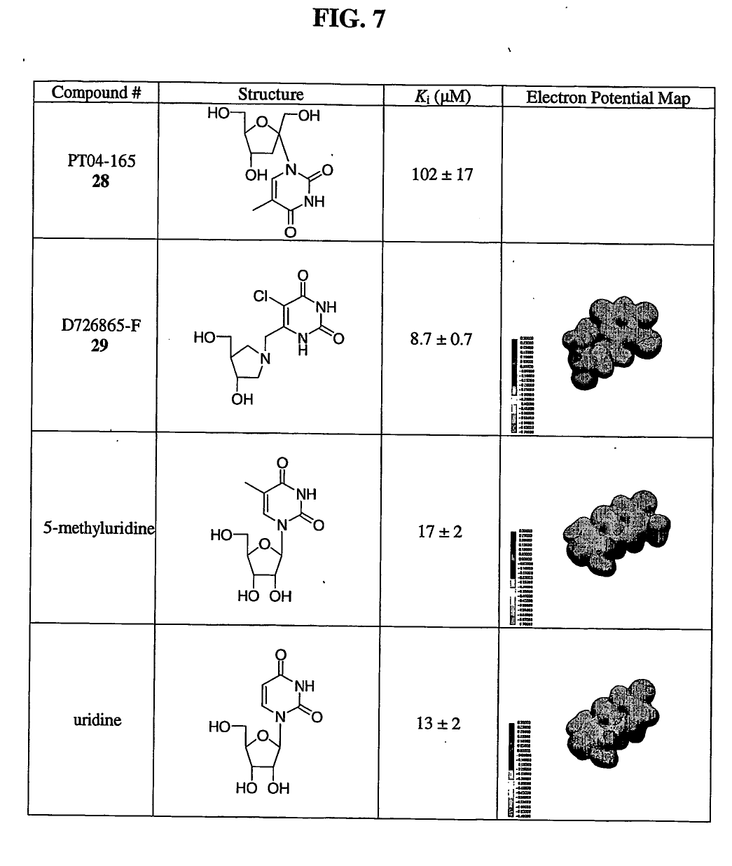

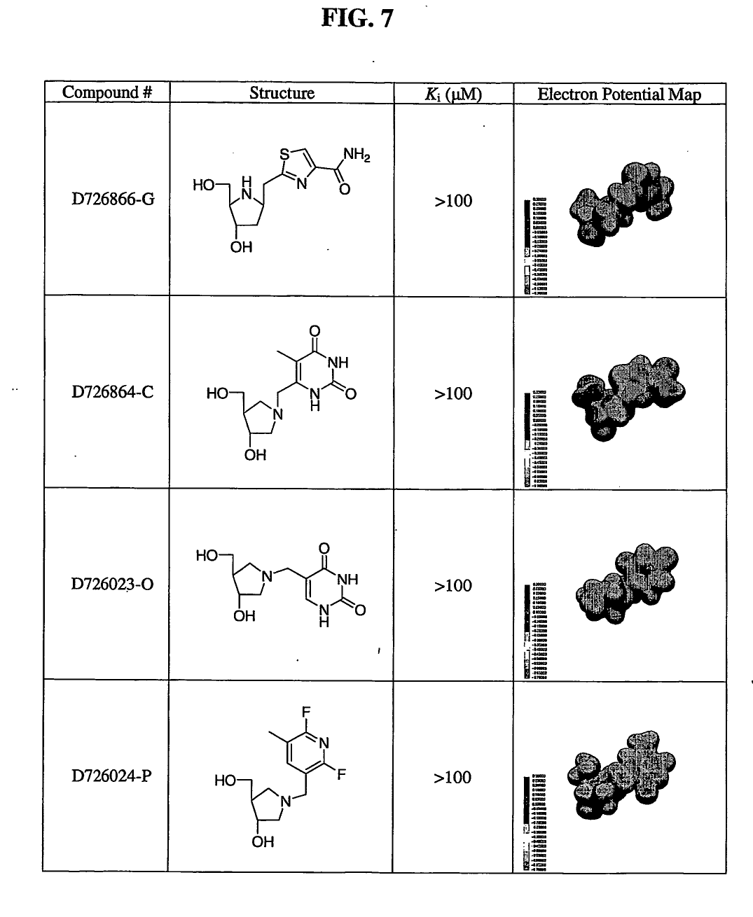

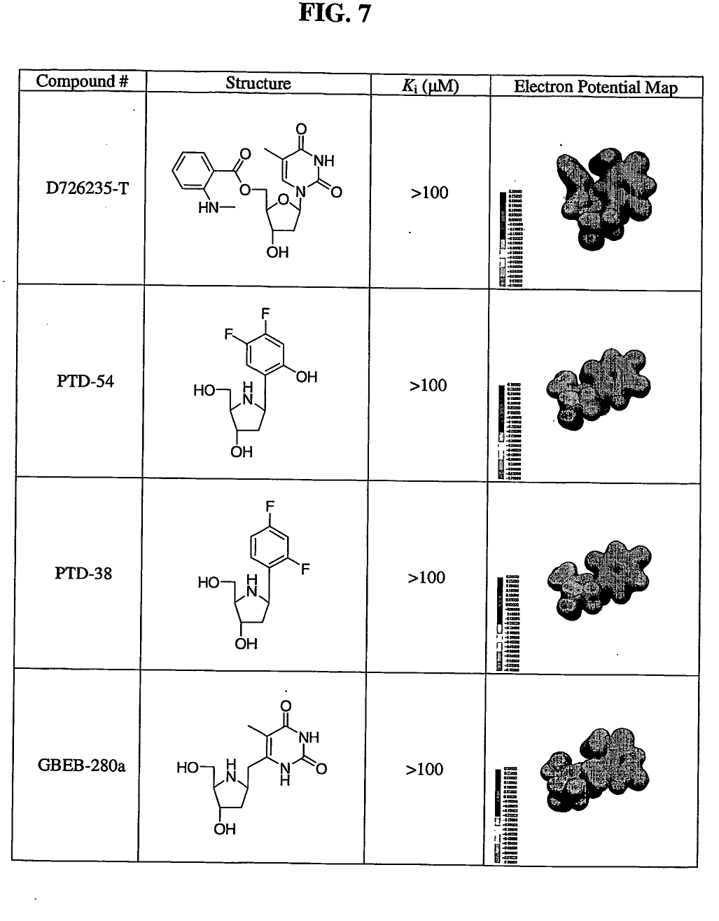

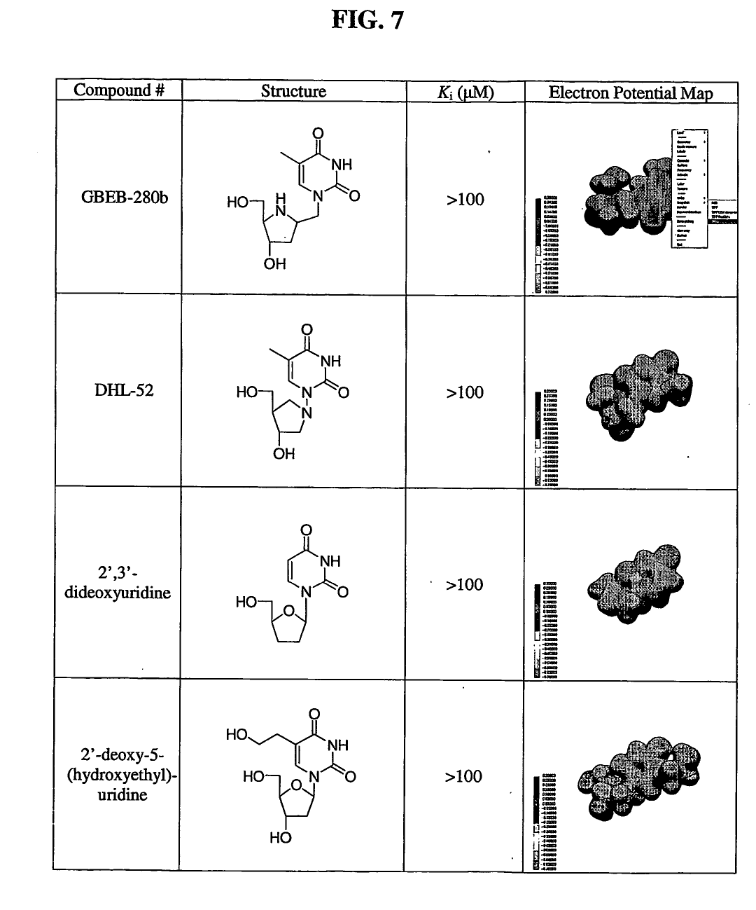

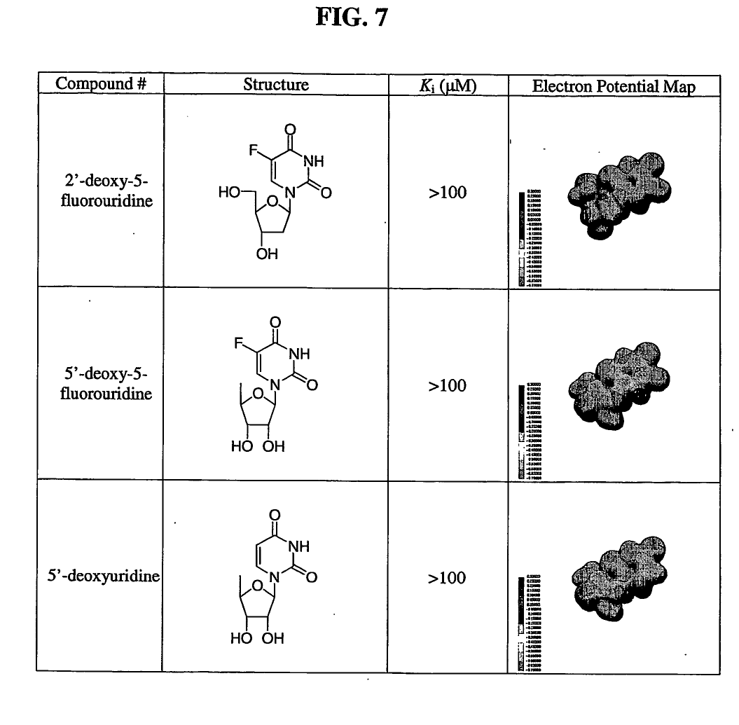

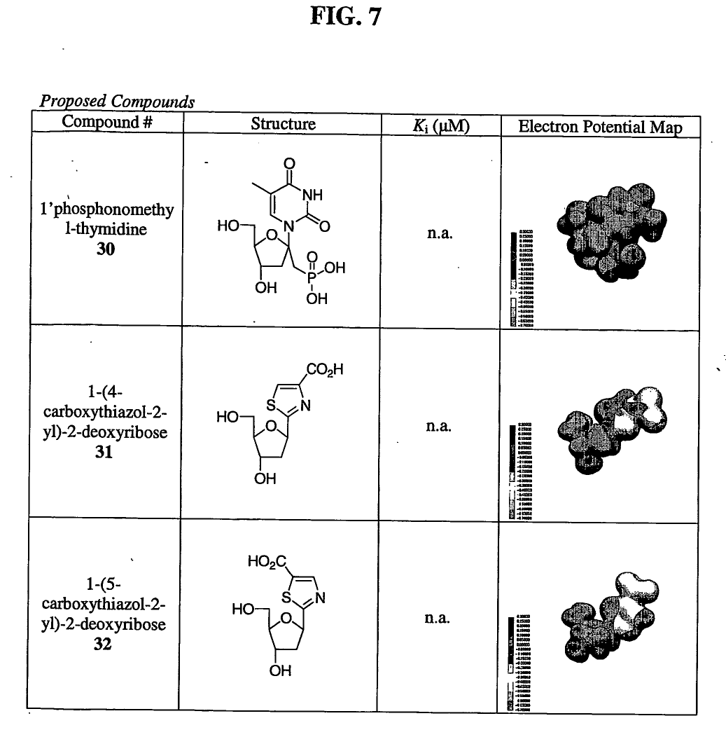

Accordingly, in some embodiments, the invention is directed to transition state inhibitors of a thymidine phosphorylase (TP). The transition state inhibitors are compounds that resemble the charge and geometry of the thymidine phosphorylase transition state. Non-limiting examples of TP transition state inhibitors are provided as compounds 1-22 of FIG. 2 and compounds 23-32 of FIG. 7. More preferred inhibitors are compounds 9-12, 23, 26, and 29, because those compounds most closely resemble the TP transition state. The skilled artisan would understand that, although the compounds of FIG. 2 are shown as the phosphonic acid, other salt forms are equally effective, and that they will exist in biological fluids and in buffered aqueous solutions as mixtures of salt and acid forms depending upon the biological fluid or buffer. It would also be understood that the inhibitor compounds of FIG. 2 and FIG. 7 may also be in the form of phosphonic acid ester derivatives and that these will constitute pro-drug forms of these compounds, capable of being converted to the active forms by ester cleavage.

As used herein, a compound resembles the charge and geometry of the TP transition state if the compound is as much alike in charge and geometry of the TP transition state as any of compounds 1-22 of FIG. 2 or compounds 23-32 of FIG. 7.

The transition state inhibitors of these embodiments would be expected to inhibit a thymidine phosphorylase from any archaeal, bacterial or mammalian species (including humans), since the enzyme is very similar structurally between kingdoms (Cole et al., 1999).

The above-described TP transition state inhibitors can be formulated in a pharmaceutically acceptable excipient, for pharmaceutical administration to a mammal, including humans. These formulations can be prepared for administration without undue experimentation for any particular application. The inhibitor compositions can also be prepared alone or in combination with other medications, such as chemotherapeutic agents. Additionally, proper dosages of the inhibitors can be determined without undue experimentation using standard dose-response protocols.

Accordingly, the inhibitor compositions designed for oral, lingual, sublingual, buccal and intrabuccal administration can be made without undue experimentation by means well known in the art, for example with an inert diluent or with an edible carrier. The compositions may be enclosed in gelatin capsules or compressed into tablets. For the purpose of oral therapeutic administration, the pharmaceutical inhibitor compositions of the present invention may be incorporated with excipients and used in the form of tablets, troches, capsules, elixirs, suspensions, syrups, wafers, chewing gums and the like.

Tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, corn starch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and nontoxic in the amounts used.

The inhibitor compositions of the present invention can easily be administered parenterally such as for example, by intravenous, intramuscular, intrathecal or subcutaneous injection, either alone or combined with another medication, e.g., a chemotherapeutic agent. Parenteral administration can be accomplished by incorporating the compositions of the present invention into a solution or suspension. Such solutions or suspensions may also include sterile diluents such as water for injection, saline solution, fixed oils, polyethylene glycols, glycerin, propylene glycol or other synthetic solvents. Parenteral formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering the pharmaceutical inhibitor compositions into the rectum or large intestine. This can be accomplished using suppositories or enemas. Suppository formulations can easily be made by methods known in the art. For example, suppository formulations can be prepared by heating glycerin to about 120° C., dissolving the composition in the glycerin, mixing the heated glycerin after which purified water may be added, and pouring the hot mixture into a suppository mold.

Transdermal administration includes percutaneous absorption of the inhibitor composition through the skin. Transdermal formulations include patches (such as the well-known nicotine patch), ointments, creams, gels, salves and the like.

The present invention includes nasally administering to the mammal a therapeutically effective amount of the inhibitor composition. As used herein, nasally administering or nasal administration includes administering the composition to the mucous membranes of the nasal passage or nasal cavity of the patient. As used herein, pharmaceutical compositions for nasal administration of a composition include therapeutically effective amounts of the composition prepared by well-known methods to be administered, for example, as a nasal spray, nasal drop, suspension, gel, ointment, cream or powder. Administration of the composition may also take place using a nasal tampon or nasal sponge.

In other embodiments, the invention is directed to methods of inhibiting a thymidine phosphorylase with transition state analogs. The methods comprise combining the thymidine phosphorylase with any transition state inhibitor that resembles the charge and geometry of the TP transition state. In some embodiments, the inhibitor is first designed to resemble the transition state and then synthesized. In preferred embodiments, the transition state inhibitor is one of compounds 1-22 of FIG. 2 or compounds 23-32 of FIG. 7, their salts, or their phosphonic acid ester derivatives. More preferably, the transition state inhibitor is one of compounds 9-12, 23, 26, or 29, their salts, or their phosphonic acid ester derivatives.

Although these methods could utilize any thymidine phosphorylase, i.e., from any bacterial, arcbaeal, or eukaryotic species, it is preferred that the thymidine phosphorylase is a mammalian, and most preferably a human thymidine phosphorylase, since the enzyme has a clear effect on mammalian angiogenesis, and is associated with human disease, in particular cancer.

Consequently, although these methods could be utilized with thymidine phosphorylases in vitro, it would be expected that the methods would have their greatest usefulness with thymidine phosphorylases in a living cell, most preferably a cell that is part of a living mammal. It is anticipated that the method would be particularly useful in humans with a disease associated with inappropriate induction of angiogenesis, for example cancer, in particular colon cancer, colorectal cancer, gastrointestinal cancer, and adenocarcinoma (Aoli et al., 2002; Tokunaga et al., 2002; Tsukagoshi et al., 2003; Sato et al., 2003), where thymidine phosphorylase activity is associated with more virulent phenotypes and/or resistance to chemotherapeutic agents that are TP substrates. Thus, in preferred embodiments, the cancer tissue expresses thymidine phosphorylase (Igawa et al., 2003). In other preferred embodiments, the human is being treated with a chemotherapeutic nucleoside substrate of the thyrnidine phosphorylase, e.g., 5-fluoro-2′-deoxyuridine.

The human in need of inhibition of thymidine phosphorylase can alternatively have a disease characterized by excessive or inappropriate angiogenesis, for example rheumatoid arthritis, retinal diseases such as diabetic retinopathy and age related macular degeneration, atherosclerosis, various diseases of female reproductive organs such as endometriosis, or obesity.

In related embodiments, the invention is directed to methods of treating cancer in a mammal. The methods comprise administering to the mammal a transition state inhibitor that resembles the charge and geometry of the TP transition state. As with previous embodiments, preferred inhibitors are any of compounds 1-22 of FIG. 2 or compounds 23-32 of FIG. 7, their salts, or their phosphonic acid ester derivatives; more preferred inhibitors are compounds 9-12, 23, 26, or 29, their salts, or their phosphonic acid ester derivatives.

As discussed in relation to embodiments described above, it is expected that this method would be particularly useful for treatment of colon cancer, colorectal cancer, gastrointestinal cancer, or adenocarcinoma, or where the cancer tissue expresses thymidine phosphorylase. Also, the treatment would be expected to enhance the effectiveness of a chemotherapeutic nucleoside substrate of the thymidine phosphorylase, e.g., 5-fluoro-2′-deoxyuridine.

The present invention is additionally directed to methods of inhibiting angiogenesis in a mammal, the methods comprise administering to the mammal a transition state inhibitor that resembles the charge and geometry of the TP transition state. As with previous embodiments, preferred inhibitors are any of compounds 1-22 of FIG. 2 and compounds 23-29 of FIG. 7, their salts, or their phosphonic acid ester derivatives; more preferred inhibitors are compounds 9-12, 23, 26, and 29, their salts, or their phosphonic acid ester derivatives.

These methods would be effective with any disease characterized by excessive or inappropriate angiogenesis, where the angiogenesis is at least partially caused by thymidine phosphorylase, for example cancer, in particular colon cancer, colorectal cancer, gastrointestinal cancer, or adenocarcinoma, particularly where the cancer tissue expresses thymidine phosphorylase. Examples of other diseases characterized by excessive or inappropriate angiogenesis are rheumatoid arthritis, diseases of the retina such as diabetic retinopathy and macular degeneration, atherosclerosis, various diseases of female reproductive organs such as endometriosis, and obesity.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### EXAMPLE 1

### Determination of the Thymidine Phosphorylase Transition State.

Introduction

Enzymes can catalyze highly unfavorable reactions by stabilizing the transition state. Thus, unreactive transition state analogues should be powerful inhibitors. For example, a transition state-analogue inhibitor of PNP binds with Kᵢ<10 μM. Determination of the transition state can be accomplished through the use of kinetic isotope effect (KIE) analysis followed by computer modeling. This Example describes the determination of the thymidine phosphorylase transition state by KIE analysis.

Materials and Methods

Synthesis of Labeled Thymidines.

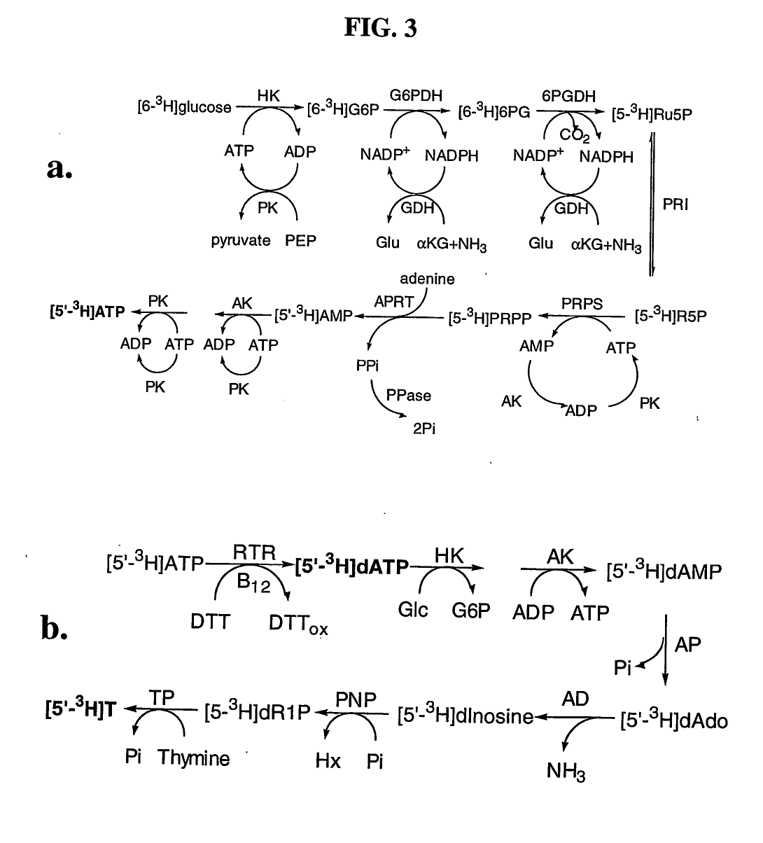

In the case of TP, radiolabeled thymidine can be enzymatically synthesized from a variety of starting materials, primarily glucose. See, for example, FIG. 3, for a method of labeling thymidine.

Determination of Kinetic Isotope Effects (KIEs).

KIEs for the radiolabeled positions were measured by mixing the label of interest with a remote label ([5′-¹⁴C]dT for ³H KIEs and [4′-³H]dT for ¹⁴C KIEs) in at least 3:1::³H:¹⁴C. Sodium arsenate and TP were added and the reaction was allowed to proceed to 20-30% completion and divided into three aliquots. Two aliquots were removed and the unreacted dT was separated from 2-deoxyribose by charcoal chromatography. The remaining aliquot was reacted to 100% completion and separated by charcoal chromatography. The eluted 2-deoxyribose was mixed with scintillation fluid and counted.

The ³H and ¹⁴C spectra were deconvoluted to give an accurate ratio of ³H to ¹⁴C in both the ˜25% and 100% reaction mixtures. The measured EKIE is defined as in Equation 1.

$${KIE}_{meas} = \frac{{( {cpm}_{light} / {cpm}_{heavy} )}_{30 \%}}{{( {cpm}_{light} / {cpm}_{heavy} )}_{100 \%}^{5}} . \qquad Equation 1$$

In the case of [1-¹⁵N]dT, HPLC was used to separate substrate from products and the ratio of isotopes was determined by electrospray ionization mass spectrometry. The [1-¹⁵N]dT KIE was calculated by observing thymine.

The [1′-¹⁴C] KIE was corrected for the KIE of the remote label used ([4′-³H]dT) and the [1-¹⁵N]dT KIE was corrected for natural abundance isotope enrichment of the ‘unlabeled’ compound. All KIEs were then corrected for isotopic depletion according to Equation 2.

$${KIE}_{actual} = ln ( 1 - {KIE}_{meas} ) ⨯ \frac{fraction converted}{ln ( 1 - fraction converted )} \qquad Equation 2$$

Results

Commitment to Catalysis.

Reaction reversibility and commitment to catalysis are typically the most important sources of KIE suppression. In this study, enzymatic reversibility was prevented by the use of arsenate as the attacking nucleophile in place of phosphate. Commitment to catalysis, which describes the tendency of bound substrate to either proceed to product or to be released into solution, was measured. High commitment to catalysis (all bound substrate proceeds to product) will suppress the experimental KIE.

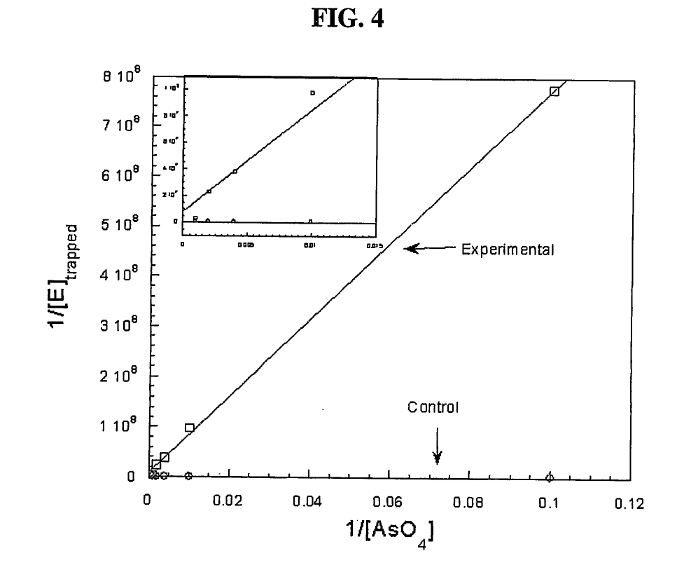

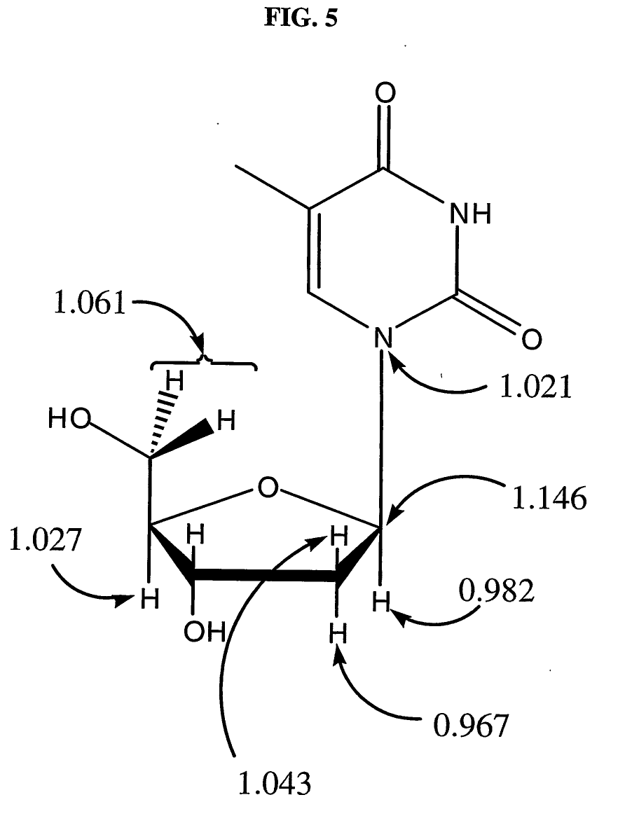

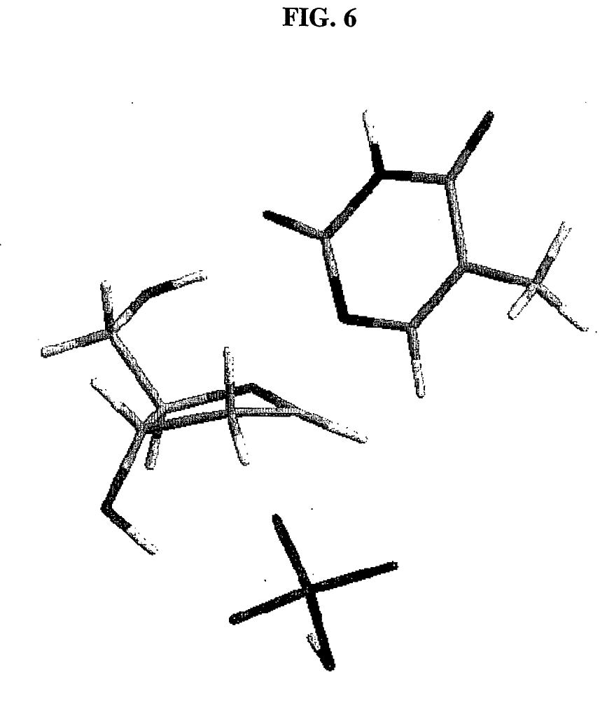

FIG. 4 shows that the arsenolysis of thymidine by TP demonstrates 0.70% commitment to catalysis at 23 μM TP, hence the experimental and intrinsic KIEs are substantially identical. The intrinsic KIEs are provided in Table 1 and FIG. 5.

TABLE 1

| Position | Intrinsic KIE |
|---|---|
| 1′-3H | 0.982 ± 0.002 |
| 1′-14C | 1.146 ± 0.005 |
| 2′R-3H | 0.967 ± 0.002 |
| 2′S-3H | 1.043 ± 0.002 |
| 4′-3H | 1.027 ± 0.003 |
| 5′-3H | 1.068 ± 0.003 |
| 5′-14C | 1.000* |
| 1-15N | 1.021 ± 0.005 |

*KIE assumed to be unity.

Transition State Calculations.

The TS structure was modeled using density functional theory (B1LYP/6-31 G*) as implemented in Gaussian 98. A starting structure was determined using the Synchronous Transit-Guided Quasi-Newton method with no constraints. Theoretical KIEs were then calculated using Isoeff98. Constraints were then added, the TS redetermined using the Berny Algorithm, and KIEs were again calculated. The constraints are varied in a systematic manner until the calculated KIEs approximately match those measured. The structure provided in FIG. 6 is considered to be the TS. Final distance constraints were between 1-N and 1′-C, 1′-C and O(phosphate), and 1′-C and 1′-H. The last constraint was added to regulate out-of-plane vibrations. The calculated and intrinsic KIEs, along with the ΔKIE for each position are provided in Table 2.

TABLE 2

| | Calculated | Intrinsic | |
|---|---|---|---|
| Position | KIE | KIE | ÄKIE |
| 1′-³H | 0.981 | 0.982 ± 0.002 | 0.001 |
| 1′-¹⁴C | 1.144 | 1.146 ± 0.005 | 0.002 |
| 2′R-³H | 0.941 | 0.967 ± 0.002 | 0.026 |
| 2′S-³H | 1.026 | 1.043 ± 0.002 | 0.017 |
| 4′-³H | 1.046 | 1.027 ± 0.003 | 0.019 |
| 5′-³H | 1.066* | 1.068 ± 0.003 | 0.002 |
| 5′-¹⁴C | 1.001 | 1.000 | 0.001 |
| 1-¹⁵N | 1.012 | 1.021 ± 0.005 | 0.009 |

*Average of 5′proR - and 5′proS-³H

### CONCLUSIONS

The transition state model provided herein provides clear evidence that the transition state is not dissociative or S_{N}1-like as previously assumed. The TS demonstrates an S_{N}2 type reaction with substantial participation of both the nucleophile and the leaving group. The above model has 0.24 and 0.22 bond order to the leaving group and nucleophile, respectively. Based on these novel results, the TS is approximately symmetric with substantial bond order to both the attacking nucleophile and the leaving group. There appears to be little oxocarbenium charge buildup. The differences in 1-N KIE are explained by some leaving group activation from active site Arg and Lys residues that were not taken into account in the calculations. Inhibitor designs based on this newly-discovered transition state structure will constitute transition state analog inhibitors.

### EXAMPLE 2

### Designing Thymidine Phosphorylase Inhibitors Using Electron Potential Mapping

Compounds shown in FIG. 7 were analyzed for ability to inhibit thymidine phosphorylase and mapped for electron potential.

Experimental:

Binding constants were determined by UV-Vis spectrophotometery using 2′-deoxy-5-nitrouridine (5NdU) as the substrate (Kₘ=50 μM at pH 6.01). The reaction mixture consisted of inhibitor (approximately 1/10×Kᵢ to 10×Kᵢ), 50 mM MES (pH 6.01), 150 mM NaCl, 1 mM EDTA, 5 mM inorganic phosphate, and 250 μM 5NdU in a total of 1 ml. Thymidine phosphorylase was added to approximately 10 nM (1 nM enzyme was used for D723333-P) and the reaction was monitored at λ=347 nm for 30 min. At least five concentrations of inhibitor were used for each compound and experiments were repeated in triplicate. Initial rates were determined by a linear least-squares fit. The Kᵢ for each compound was then determined using the following equation:

$$v = \frac{V ⨯ [ S ]}{[ S ] + {K}_{m} ( \frac{1 + [ I ]}{{K}_{ i}} )}$$

where ν is the rate of reaction and V is the maximal rate in the absence of inhibitor.

Computational:

Maps of electron potential density were constructed using Gaussian98 and Molekel v4.3. Compounds were minimized in vacuo at HF/3-21G with the pyrimidine ring, or pyrimidine analogue, constrained to a rotational angle similar to that of the calculated transition state (˜40.3° dihedral at the H1′—Cl1′—N1—C6 bond). Surfaces were then generated used Molekel's “compute electron density” function. Electronic potentials were mapped onto this surface with a scale of +0.3 (blue) to −0.7 (red).

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

What is claimed is:

1. A transition state inhibitor of a thymidine phosphorylase, wherein the transition state inhibitor is a compound that resembles the charge and geometry of the thymidine phosphorylase transition state.

2. The transition state inhibitor of claim 1, selected from the group consisting of compounds 1-22 of FIG. 2, their salts, and their phosphonic acid ester derivatives.

3. The transition state inhibitor of claim 1, selected from the group consisting of compounds 9-12 of FIG. 2, their salts, and their phosphonic acid ester derivatives.

4. The transition state inhibitor of claim 1, selected from the group consisting of compounds 23-29 of FIG. 7, their salts, and their phosphonic acid ester derivatives.

5. The transition state inhibitor of claim 1, selected from the group consisting of compounds 30-32 of FIG. 7, their salts, and their phosphonic acid ester derivatives.

6. The transition state inhibitor of claim 1, selected from the group consisting of compounds 23, 26, and 29 of FIG. 7, their salts, and their phosphonic acid ester derivatives.

7. The transition state inhibitor of claim 1, in a pharmaceutically acceptable excipient.

8. A method of inhibiting a thymidine phosphorylase, the method comprising combining the thymidine phosphorylase with the transition state inhibitor of claim 1.

9. The method of claim 8, wherein the transition state inhibitor is selected from the group consisting of compounds 1-22 of FIG. 2, their salts, and their phosphonic acid ester derivatives.

10. The method of claim 8, wherein the transition state inhibitor is selected from the group consisting of compounds 9-12 of FIG. 2, their salts, and their phosphonic acid ester derivatives.

11. The method of claim 8, wherein the transition state inhibitor is selected from the group consisting of compounds 23-29 of FIG. 7, their salts, and their phosphonic acid ester derivatives.

12. The method of claim 8, wherein the transition state inhibitor is selected from the group consisting of compounds 30-32 of FIG. 7, their salts, and their phosphonic acid ester derivatives.

13. The method of claim 8, wherein the transition state inhibitor is selected from the group consisting of compounds 23, 26, and 29 of FIG. 7, their salts, and their phosphonic acid ester derivatives.

14. The method of claim 8, wherein the thymidine phosphorylase is a mammalian thymidine phosphorylase.

15. The method of claim 8, wherein the thymidine phosphorylase is a human thymidine phosphorylase.

16. The method of claim 8, wherein the thymidine phosphorylase is in a living cell.

17. The method of claim 16, wherein the cell is part of a living mammal.

18. The method of claim 17, wherein the mammal is a human with cancer.

19. The method of claim 18, wherein the cancer is colon cancer, colorectal cancer, gastrointestinal cancer, or adenocarcinoma.

20. The method of claim 18, wherein the cancer tissue expresses thymidine phosphorylase.

21. The method of claim 18, wherein the human is being treated with a chemotherapeutic nucleoside substrate of the thymidine phosphorylase.

22. The method of claim 21, wherein the chemotherapeutic nucleoside is 5-fluoro-2′-deoxyuridine.

23. A method of treating cancer in a mammal, the method comprising administering to the mammal the transition state inhibitor of claim 1.

24. The method of claim 23, wherein the transition state inhibitor is selected from the group consisting of compounds 1-22 of FIG. 2, their salts, and their phosphonic acid ester derivatives.

25. The method of claim 23, wherein the transition state inhibitor is selected from the group consisting of compounds 9-12 of FIG. 2, their salts, and their phosphonic acid ester derivatives.

26. The method of claim 23, wherein the transition state inhibitor is selected from the group consisting of compounds 23-29 of FIG. 7, their salts, and their phosphonic acid ester derivatives.

27. The method of claim 23, wherein the transition state inhibitor is selected from the group consisting of compounds 30-32 of FIG. 7, their salts, and their phosphonic acid ester derivatives.

28. The method of claim 23, wherein the transition state inhibitor is selected from the group consisting of compounds 23, 26, and 29 of FIG. 7, their salts, and their phosphonic acid ester derivatives.

29. The method of claim 23, wherein the cancer is colon cancer, colorectal cancer, gastrointestinal cancer, or adenocarcinoma.

30. The method of claim 23, wherein the cancer tissue expresses thymidine phosphorylase.

31. The method of claim 23, wherein the mammal is a human is being treated with a chemotherapeutic nucleoside substrate of the thymidine phosphorylase.

32. The method of claim 31, wherein the chemotherapeutic nucleoside is 5-fluoro-2′-deoxyuridine.

33. A method of inhibiting angiogenesis in a mammal, the method comprising administering to the mammal the transition state inhibitor of claim 1.

34. The method of claim 33, wherein the transition state inhibitor is selected from the group consisting of compounds 1-22 of FIG. 2, their salts, and their phosphonic acid ester derivatives.

35. The method of claim 33, wherein the transition state inhibitor is selected from the group consisting of compounds 9-12 of FIG. 2, their salts, and their phosphonic acid ester derivatives.

36. The method of claim 33, wherein the transition state inhibitor is selected from the group consisting of compounds 23-29 of FIG. 7, their salts, and their phosphonic acid ester derivatives.

37. The method of claim 33, wherein the transition state inhibitor is selected from the group consisting of compounds 30-32 of FIG. 7, their salts, and their phosphonic acid ester derivatives.

38. The method of claim 33, wherein the transition state inhibitor is selected from the group consisting of compounds 23, 26, and 29 of FIG. 7, their salts, and their phosphonic acid ester derivatives.

39. The method of claim 33, wherein the mammal is a, human with cancer.

40. The method of claim 39, wherein the cancer is colon cancer, colorectal cancer, gastrointestinal cancer, or adenocarcinoma.

41. The method of claim 39, wherein the cancer tissue expresses thymidine phosphorylase.

42. The method of claim 33, wherein the mammal is a human with rheumatoid arthritis.

43. The method of claim 33, wherein the mammal is a human with a disease of the retina characterized by excessive angiogenesis.

44. The method of claim 33, wherein the mammal is a human with atherosclerosis.

45. The method of claim 33, wherein the mammal is a human with endometriosis.

46. The method of claim 33, wherein the mammal is an obese human.

47. A method of designing an inhibitor of thymidine phosphorylase, the method comprising designing a compound that resembles the charge and geometry of the thymidine phosphorylase transition state.
